(19) United States
(12) Patent Application Publication
(10) Pub. No.: US 2015/0289916 A1
(43) Pub. Date: Oct. 15, 2015

(54) BONE SCREWS HAVING MORE THAN THREE LEADS

(71) Applicant: Chester H. SHARPS, Manakin Sabot, VA (US)

(72) Inventor: Chester H. SHARPS, Manakin Sabot, VA (US)

(21) Appl. No.: 14/687,125

(22) Filed: Apr. 15, 2015

Related U.S. Application Data
(60) Provisional application No. 61/979,779, filed on Apr. 15, 2014.

(51) Int. Cl. A61B 17/86; A61B 17/70

(52) U.S. Cl. CPC A61B 17/863; A61B 17/7035; A61B 17/7032

(57) ABSTRACT

A bone screw assembly, including: a bone screw having a head portion and a thread portion, wherein the thread portion includes an axial shaft and one of a quad lead thread or a penta lead thread disposed about the axial shaft; and, optionally, a rod retention member selectively coupled to the head portion of the bone screw. Optionally, the lead thread includes leads that originate at different points along the axial shaft. Optionally, the lead thread includes leads that terminate at different points along the axial shaft. Optionally, the axial shaft includes a first tapering portion proximate a tip of the axial shaft. Optionally, the axial shaft includes a second tapering portion proximate the tip of the axial shaft. Preferably, the second tapering portion tapers more steeply than the first tapering portion. Optionally, the axial shaft includes a reduced diameter portion proximate the head portion. Optionally, the axial shaft includes one or more tapering portions proximate the head portion.

### CROSS-REFERENCE TO RELATED APPLICATION

The present patent application/patent claims the benefit of priority of co-pending U.S. Provisional Patent Application No. 61/979,779, filed on Apr. 15, 2014, and entitled “BONE SCREWS HAVING MORE THAN THREE LEADS,” the contents of which are incorporated in full by reference herein.

### FIELD OF THE INVENTION

The present invention relates generally to bone screws having more than three leads, such as quad and penta lead bone screws, for use in surgical applications. More specifically, the present invention relates to bone screws having more than three leads, such as quad and penta lead bone screws, for use in spinal surgical applications, such as monoaxial and polyaxial pedicle screw applications and the like.

### BACKGROUND OF THE INVENTION

Quad and penta lead screws, for example, are used in a variety of applications. These screws utilize four or five thread starts/leads between the distal end (i.e. tip) of the screw and the proximal end (i.e. head) of the screw. The four or five thread starts are typically spaced apart by about 70-90 degrees. The screws may used to engage bone, or, more often, the quad or penta lead portion is used to engage an anatomical plate or other implant structure. The advantage of quad or penta lead screws relative to single, double, or triple lead screws is that quad or penta lead screws are advanced farther with fewer turns, i.e. they are placed more quickly and with less effort. The disadvantage of quad or penta lead screws relative to single, double, or triple lead screws is that quad or penta lead screws typically have a lower pullout or backout strength, i.e. they may become dislodged more easily and with less effort.

Thus, what is still needed in the art is a bone screw having more than three leads, such as a quad or penta lead bone screw, or the like that overcomes the above mentioned disadvantage(s).

### BRIEF SUMMARY OF THE INVENTION

In various exemplary embodiments, the present invention provides a quad or penta lead pedicle screw or the like that is capable of being placed quickly with minimal effort and that resists pullout or backout at least as effectively as comparable single, double, or triple lead screws. These quad and penta lead bone screws are ideally suited for the surgical correction of spinal deformities and other similar surgical procedures in which multiple screws must be placed. In such surgical procedures, a surgeon may be come fatigued and time is of the essence.

In one exemplary embodiment, the present invention provides a pedicle screw assembly, including: a bone screw having a head portion and a thread portion, wherein the thread portion includes an axial shaft and one of a quad lead thread or a penta lead thread disposed about the axial shaft. In a polyaxial application, the pedicle screw assembly may also include a rod retention member selectively coupled to the head portion of the bone screw. Optionally, the quad lead thread of the thread portion of the bone screw includes leads that start with about 90 degrees of separation. Optionally, the penta lead thread of the thread portion of the bone screw includes leads that start with about 72 degrees of separation. Optionally, the lead thread of the thread portion of the bone screw includes leads that originate at different points along the axial shaft of the thread portion of the bone screw. Optionally, the lead thread of the thread portion of the bone screw includes leads that terminate at different points along the axial shaft of the thread portion of the bone screw. Optionally, the axial shaft of the thread portion of the bone screw includes a first tapering portion proximate a tip of the axial shaft of the thread portion of the bone screw. Optionally, the axial shaft of the thread portion of the bone screw includes a second tapering portion proximate the tip of the axial shaft of the thread portion of the bone screw. Preferably, the second tapering portion tapers more steeply than the first tapering portion. Optionally, the axial shaft of the thread portion of the bone screw includes a reduced diameter portion proximate the head portion of the bone screw. Optionally, the axial shaft of the thread portion of the bone screw includes one or more tapering portions proximate the head portion of the bone screw. Preferably, the rod retention member is selectively pivotably coupled to the head portion of the bone screw.

In another exemplary embodiment, the present invention provides a bone screw, including: a head portion; and a thread portion, wherein the thread portion includes an axial shaft and one of a quad lead thread or a penta lead thread disposed about the axial shaft. The bone screw may also include a rod retention member selectively coupled to the head portion of the bone screw. Optionally, the quad lead thread of the thread portion of the bone screw includes leads that start with about 90 degrees of separation. Optionally, the penta lead thread of the thread portion of the bone screw includes leads that start with about 72 degrees of separation. Optionally, the lead thread of the thread portion of the bone screw includes leads that originate at different points along the axial shaft of the thread portion of the bone screw. Optionally, the lead thread of the thread portion of the bone screw includes leads that terminate at different points along the axial shaft of the thread portion of the bone screw. Optionally, the axial shaft of the thread portion of the bone screw includes a first tapering portion proximate a tip of the axial shaft of the thread portion of the bone screw. Optionally, the axial shaft of the thread portion of the bone screw includes a second tapering portion proximate the tip of the axial shaft of the thread portion of the bone screw. Preferably, the second tapering portion tapers more steeply than the first tapering portion. Optionally, the axial shaft of the thread portion of the bone screw includes a reduced diameter portion proximate the head portion of the bone screw. Optionally, the axial shaft of the thread portion of the bone screw includes one or more tapering portions proximate the head portion of the bone screw. Preferably, the rod retention member is selectively pivotably coupled to the head portion of the bone screw.

In a further exemplary embodiment, the present invention provides a bone screw, including: a head portion; and a thread portion, wherein the thread portion includes an axial shaft and one of a quad lead thread or a penta lead thread disposed about the axial shaft, wherein the axial shaft of the thread portion of the bone screw includes a first tapering portion proximate a tip of the axial shaft of the thread portion of the bone screw, and wherein the axial shaft of the thread portion of the bone screw includes a second tapering portion proximate the tip of the axial shaft of the thread portion of the bone screw, wherein the second tapering portion tapers more steeply than the first tapering portion. Optionally, the lead thread of the thread portion of the bone screw includes leads that originate at different points along the axial shaft of the thread portion of the bone screw. Optionally, the lead thread of the thread portion of the bone screw includes leads that terminate at different points along the axial shaft of the thread portion of the bone screw. Optionally, the axial shaft of the thread portion of the bone screw includes a reduced diameter portion proximate the head portion of the bone screw. Optionally, the axial shaft of the thread portion of the bone screw includes one or more tapering portions proximate the head portion of the bone screw.

### BRIEF DESCRIPTION OF THE DRAWINGS

The present invention is illustrated and described herein with reference to the various drawings, in which like reference numbers are used to denote like screw components, as appropriate, and in which:

FIG. 1 is a perspective view of one exemplary embodiment of the quad lead bone screw of the present invention;

FIG. 2 is a set of planar side views of the quad lead bone screw of FIG. 1;

FIG. 3 is a planar end (i.e. tip) view of the quad lead bone screw of FIG. 1;

FIG. 4 is a planar end (i.e. head) view of the quad lead bone screw of FIG. 1;

FIG. 5 is a perspective view of one exemplary embodiment of the penta lead bone screw of the present invention;

FIG. 6 is a set of planar side views of the penta lead bone screw of FIG. 5;

FIG. 7 is a planar end (i.e. tip) view of the penta lead bone screw of FIG. 5;

FIG. 8 is a planar end (i.e. head) view of the penta lead bone screw of FIG. 5; and

FIG. 9 is a perspective view of one exemplary embodiment of a rod retention member that may be used in conjunction with any of the bone screws of the present invention.

### DETAILED DESCRIPTION OF THE INVENTION

Again, in various exemplary embodiments, the present invention provides a quad or penta lead pedicle screw or the like that is capable of being placed quickly with minimal effort and that resists pullout or backout at least as effectively as comparable single, double, or triple lead screws. This bone screw is ideally suited for the surgical correction of spinal deformities/anomalies/trauma and other similar surgical procedures in which multiple screws must be placed. In such surgical procedures, a surgeon may be come fatigued and operative time is of the essence.

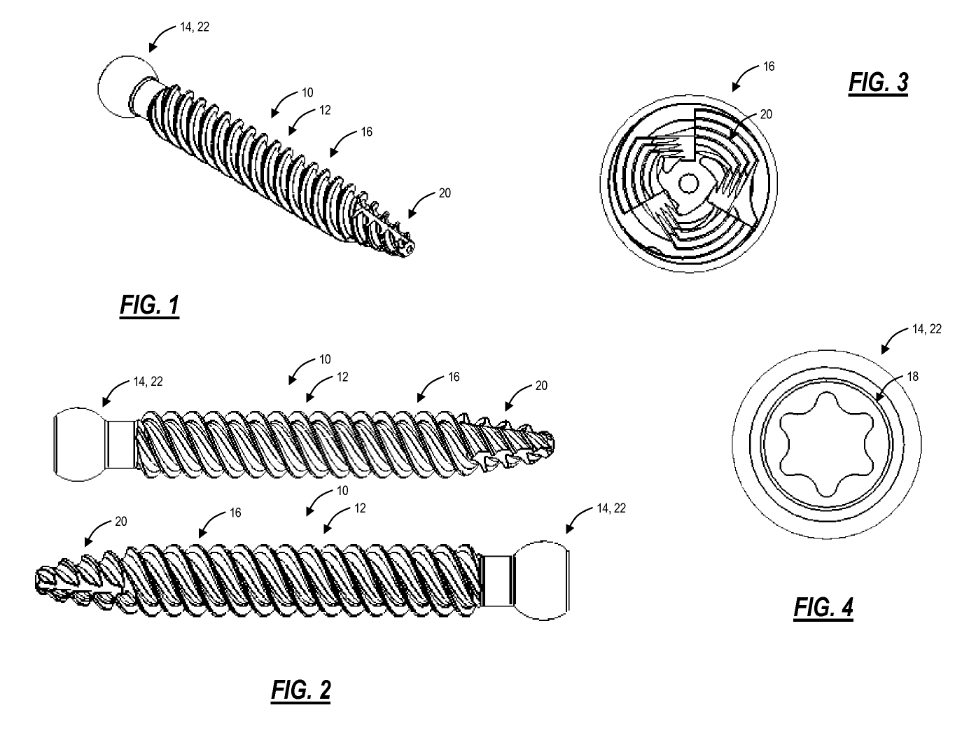

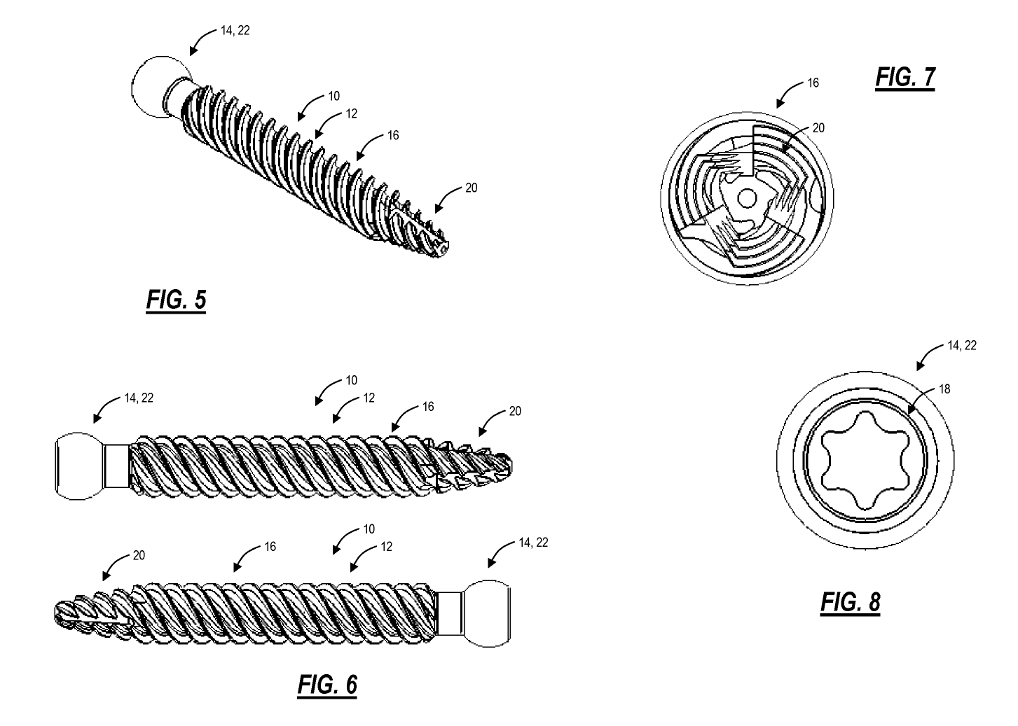

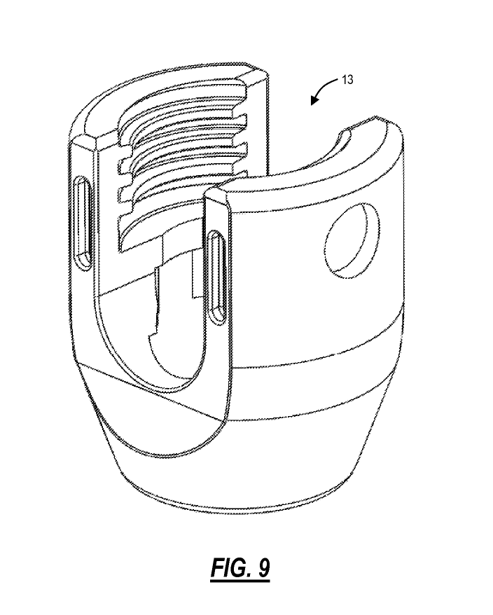

Referring to FIGS. 1, 2, 5, and 6, in two exemplary embodiments, the multi lead bone screw assembly 10 of the present invention includes a multi lead bone screw 12 and, optionally a rod retention member 13 (FIG. 9) or the like.

The multi lead bone screw 12 includes a head portion 14 and a threaded portion 16 (see also FIGS. 3,4, 7, and 8). The head portion 14 of the multi lead bone screw 12 consists of an enlarged structure, such as a wholly or partially spherical structure, a wholly or partially cylindrical structure, or the like, having one or more tool receiving recesses 18 manufactured into the top surface thereof, by which the multi lead bone screw 12 is grasped and/or turned. Optionally, six such tool receiving recesses 18 are utilized and are spaced apart by about 60 degrees, although it will be readily apparent to those of ordinary skill in the art that other suitable configurations may be utilized. The threaded portion 16 of the multi lead bone screw 12 consists of four or five thread starts/leads between the distal end (i.e. tip) 20 of the screw 12 and the proximal end (i.e. head) 22 of the screw 12. Optionally, the four or five thread starts are spaced apart by about 70-90 degrees, although it will be readily apparent to those of ordinary skill in the art that other suitable configurations may be utilized. Optionally, the four or five thread starts/leads also originate and/or terminate at different positions along the axial length of the screw 12. In other words, the four or five thread starts/leads may or may not coincide along the axial length of the screw 12 and may be spaced apart by any desired constant or variable distance. Finally, one or more of the four or five thread starts/leads may be selectively broken or notched to create a biting surface, such as near the tip 20 of the screw 12 so that the screw will advance into bone from the first turn. Any suitable thickness, pitch, or other dimensions may be utilized, as well as any suitable materials (e.g. a biocompatible metal, ceramic, or plastic, bony material, etc.).

Referring to FIG. 9, the rod retention member 13, when used, consists of a U-shaped member having a hole in the bottom. The threaded portion 16 of the bone screw 12 is disposed through this hole, with the head portion 14 of the bone screw being pivotably seated in the interior bottom of the U-shaped member, prior to the screw 12 being driven into bone. The rod retention member 13 includes a plurality of internally threaded, concentrically arranged, vertically extending arm members between which a stabilizing rod or other similar stabilizing structure (not illustrated) is selectively disposed. This stabilizing rod is then “locked” into place via the engagement of an externally threaded cap (not illustrated) into the internal threads of the arm members, thereby “locking” the orientation of the rod retention member 13 with respect to the head portion 14 of the multi lead bone screw 12, as the stabilizing rod is in direct or indirect contact with the head portion 14 of the multi lead bone screw 12. It will be readily apparent to those of ordinary skill in the art that various other structures may also be disposed between the stabilizing rod and either or both of the externally threaded cap and the head portion 14 of the multi lead bone screw 12. In this manner, screws 12 may be placed in different anatomical structures, or different parts of the same anatomical structure, and securely joined by stabilizing structures in a predetermined configuration and alignment.

The threaded portion 16 of the screw 12 includes an axial shaft that may have a non-tapering portion, a first tapering portion, and a second tapering portion disposed, in order, between the head portion 22 of the screw 12 and the rounded or pointed tip 20 of the screw 12. Preferably, the taper of the second tapering portion is greater than the taper of the first tapering portion, with the second tapering portion sweeping a 45-degree arc, a 60-degree arc, etc. The threads are disposed about these various segments of the screw 12. In one exemplary embodiment, a reduced diameter portion and one or more tapering portions are also disposed along the axial shaft between the head portion 14 and the threaded portion 16 of the screw 12.

Axial pullout testing of the triple lead bone screw 12 of the present invention demonstrated performance comparable to that of a conventional single lead bone screw.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

1. A pedicle screw assembly, comprising:
a bone screw comprising a head portion and a thread portion, wherein the thread portion comprises an axial shaft and one of a quad lead thread or a penta lead thread disposed about the axial shaft.

2. The pedicle screw assembly of claim 1, wherein the lead thread of the thread portion of the bone screw comprises leads that start with about 70-90 degrees of separation.

3. The pedicle screw assembly of claim 1, wherein the lead thread of the thread portion of the bone screw comprises leads that originate at different points along the axial shaft of the thread portion of the bone screw.

4. The pedicle screw assembly of claim 1, wherein the lead thread of the thread portion of the bone screw comprises leads that terminate at different points along the axial shaft of the thread portion of the bone screw.

5. The pedicle screw assembly of claim 1, wherein the axial shaft of the thread portion of the bone screw comprises a first tapering portion proximate a tip of the axial shaft of the thread portion of the bone screw.

6. The pedicle screw assembly of claim 5, wherein the axial shaft of the thread portion of the bone screw comprises a second tapering portion proximate the tip of the axial shaft of the thread portion of the bone screw.

7. The pedicle screw assembly of claim 6, wherein the second tapering portion tapers more steeply than the first tapering portion.

8. The pedicle screw assembly of claim 1, wherein the axial shaft of the thread portion of the bone screw comprises a reduced diameter portion proximate the head portion of the bone screw.

9. The pedicle screw assembly of claim 1, wherein the axial shaft of the thread portion of the bone screw comprises one or more tapering portions proximate the head portion of the bone screw.

10. The pedicle screw assembly of claim 1, wherein further comprising a rod retention member selectively coupled to the head portion of the bone screw, wherein the rod retention member is selectively pivotably coupled to the head portion of the bone screw.

11. A bone screw, comprising:
a head portion; and
a thread portion, wherein the thread portion comprises an axial shaft and one of a quad lead thread or a penta lead thread disposed about the axial shaft.

12. The bone screw of claim 11, wherein the lead thread of the thread portion of the bone screw comprises leads that start with about 70-90 degrees of separation.

13. The bone screw of claim 11, wherein the lead thread of the thread portion of the bone screw comprises leads that originate at different points along the axial shaft of the thread portion of the bone screw.

14. The bone screw of claim 11, wherein the lead thread of the thread portion of the bone screw comprises leads that terminate at different points along the axial shaft of the thread portion of the bone screw.

15. The bone screw of claim 11, wherein the axial shaft of the thread portion of the bone screw comprises a first tapering portion proximate a tip of the axial shaft of the thread portion of the bone screw.

16. The bone screw of claim 15, wherein the axial shaft of the thread portion of the bone screw comprises a second tapering portion proximate the tip of the axial shaft of the thread portion of the bone screw.

17. The bone screw of claim 16, wherein the second tapering portion tapers more steeply than the first tapering portion.

18. The bone screw of claim 11, wherein the axial shaft of the thread portion of the bone screw comprises a reduced diameter portion proximate the head portion of the bone screw.

19. The bone screw of claim 11, wherein the axial shaft of the thread portion of the bone screw comprises one or more tapering portions proximate the head portion of the bone screw.

20. A bone screw, comprising:
a head portion; and
a thread portion, wherein the thread portion comprises an axial shaft and one of a quad lead thread or a penta lead thread disposed about the axial shaft, wherein the axial shaft of the thread portion of the bone screw comprises a first tapering portion proximate a tip of the axial shaft of the thread portion of the bone screw, and wherein the axial shaft of the thread portion of the bone screw comprises a second tapering portion proximate the tip of the axial shaft of the thread portion of the bone screw, wherein the second tapering portion tapers more steeply than the first tapering portion.

21. The bone screw of claim 20, wherein the lead thread of the thread portion of the bone screw comprises leads that originate at different points along the axial shaft of the thread portion of the bone screw.

22. The bone screw of claim 20, wherein the lead thread of the thread portion of the bone screw comprises leads that terminate at different points along the axial shaft of the thread portion of the bone screw.

23. The bone screw of claim 20, wherein the axial shaft of the thread portion of the bone screw comprises a reduced diameter portion proximate the head portion of the bone screw.

24. The bone screw of claim 20, wherein the axial shaft of the thread portion of the bone screw comprises one or more tapering portions proximate the head portion of the bone screw.
